(19) United States
(12) Patent Application Publication
(10) Pub. No.: US 2024/0273633 A1
(43) Pub. Date: Aug. 15, 2024

(54) SYSTEM AND METHOD FOR DISABILITY BENEFITS APPLICATION

(71) Applicant: Allison BERGMANN, Eureka, MO (US)

(72) Inventor: Allison BERGMANN, Eureka, MO (US)

(21) Appl. No.: 18/443,152

(22) Filed: Feb. 15, 2024

Related U.S. Application Data
(60) Provisional application No. 63/445,996, filed on Feb. 15, 2023.

(51) Int. Cl. G06Q 40/08; G06F 9/451; G06F 40/106; G06F 40/166; G16H 10/20; G16H 10/60; G16H 50/70

(52) U.S. Cl. CPC G06Q 40/08; G06F 9/451; G06F 40/106; G06F 40/166; G16H 10/20; G16H 10/60; G16H 50/70

(57) ABSTRACT

One or more techniques and/or systems are disclosed for disability benefits analysis and submission. Inputs are received relating to a disability benefits application to be submitted and processed to generate application data for a disability benefits application. Historical data relating to the plurality of previously submitted disability benefits applications is accessed and the application data is updated based on the historical data to generate updated application data, wherein the updated application data has a greater likelihood of approval than the application data. A disability benefits application using the updated application data is generated.

### CROSS-REFERENCE TO RELATED APPLICATIONS

This non-provisional application claims the benefit of and priority to U.S. Provisional Application No. 63/445,996, filed Feb. 15, 2023. The entire disclosure of the prior application is hereby incorporated by reference herein in its entirety.

### BACKGROUND

Systems for electronically submitting different applications often involve much user input without much guidance. And in some instances, such as for filing for or requesting certain benefits, the application process is manual or semi-automated. However, it is often difficult to determine whether the submission is likely to be approved and what could be done to increase the likelihood of approval and/or reducing the number of iterations or supplements needed to have the application completely processed. And still sometimes, manual interactions are needed.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems described herein can be utilized for facilitating submission of requests or applications for different benefits, particularly disability benefits. For example, various implementations provide one or more systems and interfaces to simplify the application process and increase the likelihood of a successful application approval, as well as improve the overall user experience.

In one implementation, a system for generating a disability benefits application comprises a data storage device storing historical data relating to a plurality of previously submitted disability benefits applications and a processor. The system further comprises a non-transitory computer-readable medium storing instructions that are operative upon execution by the processor to receive inputs relating to a disability benefits application to be submitted; process the inputs to generate application data for a disability benefits application; access the historical data relating to the plurality of previously submitted disability benefits applications; update the application data based on the historical data to generate updated application data, wherein the updated application data has a greater likelihood of approval than the application data; generate a disability benefits application using the updated application data; and transmit the disability benefits application to a disability application system for processing disability benefits.

In one implementation, a computerized method for generating a disability benefits application comprises receiving inputs relating to a disability benefits application to be submitted; processing the inputs to generate application data for a disability benefits application; accessing the historical data relating to the plurality of previously submitted disability benefits applications; updating the application data based on the historical data to generate updated application data; and generating via a user interface, a graphical representation including the updated application data, the updated application data comprising data having a greater likelihood of approval than the application data.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 is a block diagram illustrating a system having a disability benefits application analyzer according to an implementation.

FIG. 2 illustrates a process flow according to various implementations.

FIGS. 3-23 illustrate user interfaces according to various implementations.

FIG. 24 illustrates an application analysis and review process according to various implementations.

FIGS. 25-34 illustrate user interfaces according to various implementations.

FIG. 35 is a block diagram of an example computing environment suitable for implementing various examples of the process flow and user interfaces.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The methods and systems disclosed herein, for example, may be suitable for use in generating and submitting different applications, such as applications for disability benefits. One or more implementations facilitate entry, review, analysis, and submission of the applications in an automated process that reduce or eliminate repeated steps, such as reducing the number of iterations or supplements needed to have the application completely processed, as well as identifying possible changes to the inputs, thereby reducing errors in the application process while improving overall accuracy of data and/or increasing the likelihood of approval. User interfaces including graphical representations facilitate guided input of data that improves user efficiency via the user interface interaction and improves user performance via the user interface. For example, user interface features, such as voice assistant and auto-filling application with voice responses, improve accessibility for disabled audiences, reduce effort, and decrease time.

In some examples, received inputs relating to disability benefits are processed to increase the likelihood of approval of the disability benefits, with a disability benefits submission generated using the processed inputs that results in enhanced reliability of submission data. By reducing the number of iterations or supplements needed to have the application completely processed, the functioning of the underlying computing device is thereby improved, which reduces system resource usage.

Further, aspects of the disclosure improve the usability of the data and/or the underlying device at least by generating more relevant or complete data. Efficiency is also improved via display of the data, and user interaction performance is also improved via the user interfaces as described herein. This overall improves the human machine interaction.

In some examples, a website and a platform are provided, wherein an external-facing website allows access by users and an account-based portal or platform allows users (e.g., customers) to log into (after creating an account) and where the users apply for disability benefits. In one or more examples, the website and platform are configured to provide “BenefitsBoost services” having an improved interaction performance.

In one or more implementations, a user is guided through the application process, and once a reviewed and approved application is completed, the application is submitted to a reviewing organization (e.g., the social security administration (SSA) or a disability benefits organization), which is some examples includes using an application programming interface (API) connection.

Thus, various examples provide an efficient and user-friendly process for disability benefits submission. In this manner, when a processor is programmed to perform the operations described herein, the processor allows for more efficient and accurate disability benefits submission, which results in an improved user (e.g., customer) experience and improved functioning of the underlying computing device.

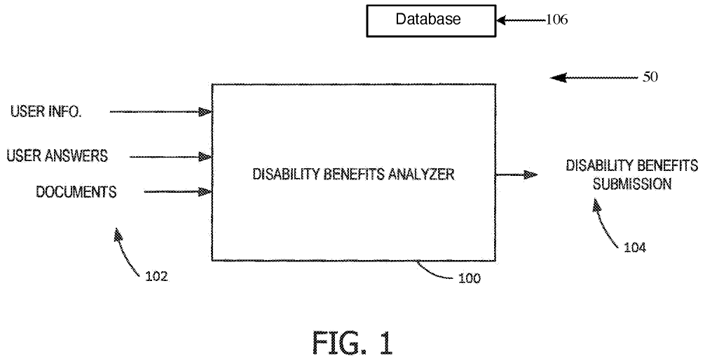

As one example, a system 50, which in some examples is configured as an operating platform or portal, includes a disability benefits application analyzer 100 as illustrated in FIG. 1. The disability benefits application analyzer 100 performs or implements one or more processes as described herein. The disability benefits application analyzer 100 is configured to receive one or more inputs 102, such as different user information, user's answers to questions, uploaded documents, etc. which can be any inputs that are useful or that can affect the disability benefits application process. Additionally, one or more variables, criteria, etc. can be inputs to the disability benefits application analyzer 100. In some examples, one or more user interfaces facilitate entry of the inputs 102 in a guided environment. It should be noted that various examples are capable of being implemented in different applications or environments, such as business-to-consumer (B2C) or business-to-business (B2B) applications.

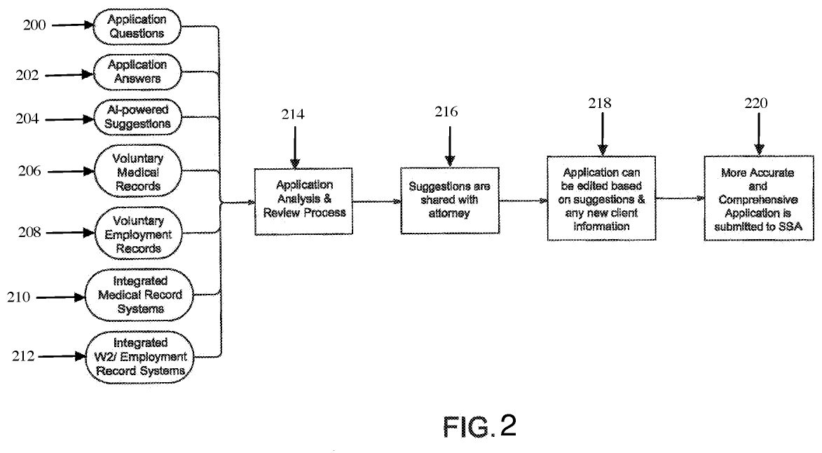

In some examples, the inputs 102 include or are based on one or more of application questions 200, application answers 202, artificial intelligence (AI)-powered suggestions 204, voluntary medical records 206, voluntary employment records 208, data from integrated medical record systems 210, data from an integrated W2/employment records systems 212, etc. as illustrated in FIG. 2. It should be appreciated that when reference is made to “integral”, in some examples this refers to the data being accessible or having an interface to the disability benefits application analyzer 100.

In various examples, the disability benefits application analyzer 100 processes the inputs 102 to generate a disability benefits submission 104, which can be submitted, for example, to the SSA. The generated disability benefits submission 104 can be different types of submissions, such as an application for disability benefits. The disability benefits application analyzer 100 processes the received inputs 102 to analyze the data and identify potential issues (such as using correlation or other analysis), provide suggestions, identify additional information needed, etc. As such, once the one or more inputs 102 are analyzed, the generated disability benefits submission 104 in various examples is an improved output that increases the likelihood of approval for disability benefits. In various examples, the disability benefits application analyzer 100 allows for a personalized and individualized user experience that leverages know-how or other data for disability benefits submission. In some examples, a database 106 (e.g., a database of historical data relating to previously submitted applications) is updated (such as using artificial intelligence or machine learning) to increase the likelihood the users receive the applied for benefits. For example, the database 106 is a dynamic database that is periodically or continuously updated based on the analysis of applications, identification of issues in the applications, etc. In some examples, the disability benefits application analyzer 100 is a disability benefits initial application analyzer. However, the disability benefits application analyzer 100 is operable at different points of the application process, appeal process, etc.

In some examples, the inputs 102 are provided to an application analysis and review process at 214 that identifies and generates suggestions that are provided to, for example, an attorney at 216. The application in some examples can be edited or updated based on the suggestions and any new information (e.g., new client health information) at 218. As described in more detail herein, the suggestions are generated based on information analyzed from other applications, such as applicants having similar health conditions, requesting similar benefits, etc. As a result, a more accurate and comprehensive application is generated and submitted at 220. That is, using stored knowledge of past applications, and providing suggestions to a current application, a likelihood of successful submission and/or approval is increased.

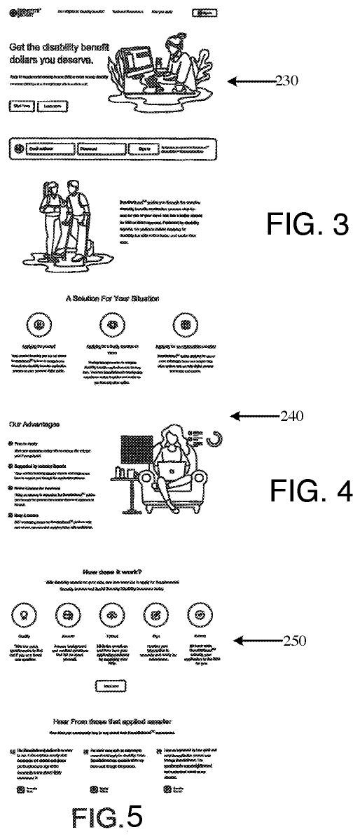

In various examples, an external-facing website (e.g., user facing website) is configured to operate with an account-based platform or portal to which users (e.g., consumers) login to complete and submit an application, such as a disability benefits application. For example, as illustrated in FIGS. 3-5, user interface 230, 240, 250 include graphical representations introducing the herein described systems. As will now be described, one or more user interfaces are configured to acquired data (e.g., user input data) that is processed by the disability benefits application analyzer 100, one or more individuals, etc. In some examples, a representative acting on behalf of the applicant can facilitate submitting the application to the SSA on the individual's behalf as their representative through the initial application process. In some examples, the submission is performed using an interface or other connection between the herein described examples and the SSA system or other system.

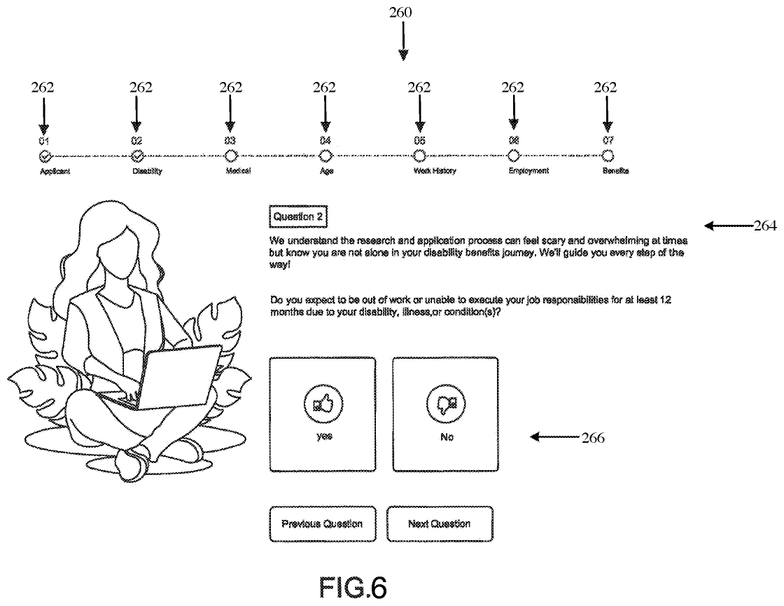

As can be seen in FIG. 6., a timeline 260 is displayed as a graphical representation of progress of the eligibility process in some examples. The timeline identifies different points 262 along the eligibility determination process and provides an indication of the completion of each of the points, such as with a check mark or other indicator of completion. FIG. 6 in the illustrated example relates to an eligibility questionnaire determining if an individual meets minimum requirements and is a suitable candidate to apply for disability benefits. The timeline 260 in this example shows the progress of the eligibility questionnaire (not the application). However, the timeline 260 can be modified or changes to represent different points along other desired or required processes.

For example, after an eligibility questionnaire is completed, the Applicant and Disability points 262 are indicated as completed as can be seen in FIG. 6. In some examples, the eligibility questionnaire is generated and displayed as part of a user interface to determine whether the user (e.g., applicant) meets certain minimum criteria, such as for SSI or SSDI disability benefits. In one example, the questionnaire is arranged having a number of inputs (e.g., user input fields) that present the minimum criteria for one or more benefits (e.g., based on user input information on medical condition, etc.) in a manner that non-experts can easily understand and answer in a format that facilitates engagement and participation until the end of the application process. A sample question 264 and graphical representations 266 that facilitate the process are shown. However, it should be appreciated that other text and/or graphical representations can be presented (e.g., displayed to the user). It should also be appreciated that different types of questions can be asked, such as related to the type of disability benefit, an expected time to be out of work, whether a physician has been seen, the user's age, whether the user has worked or is currently working, previous application for disability benefits, etc. One set of sample questions includes the following:
Question 1: Are you interested in disability benefits or are you taking this questionnaire for someone else?
Question 2: Do you expect to be out work for at least 12 months due to your disability?
Question 3: Have you seen at least one physician for your disability?
Question 4: What is your age?′
Question 5: Have you worked at least (minimum years working requirement for SSDI based on age set forth by the SSAJ)?
Question 5.2: For those that have not worked: Is that because you are a parent, child, spouse, or former spouse of the individual that has or had the disability?
Question 6: Are you currently working?
Question 6.2: Are you making $1,200 or more per month?
Question 7: Have you ever applied for disability benefits?
Question 7.2: Have you been denied for disability benefits within 60 days?

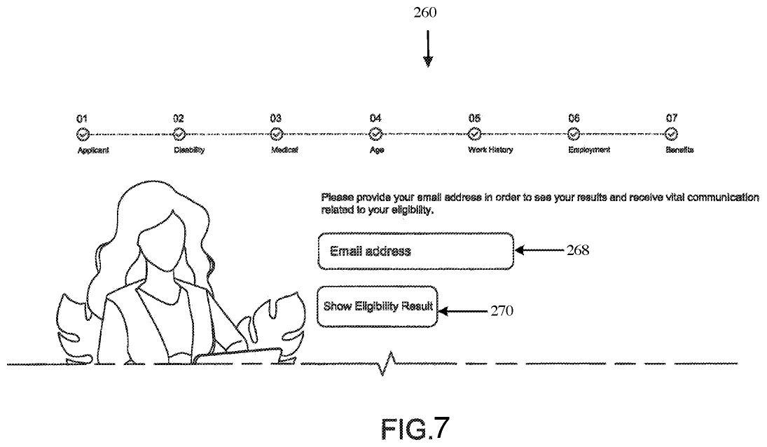

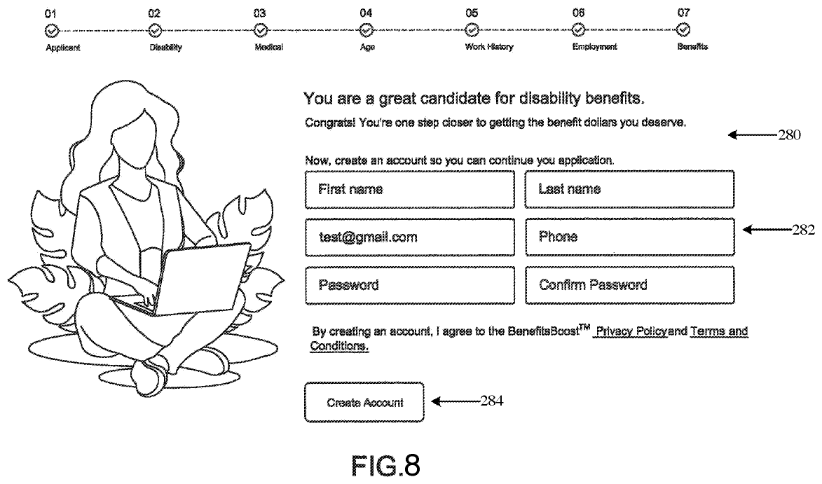

In various examples, one or more inputs are obtained using different user interfaces. For example, one or more user interfaces are configured to receive user inputs relating to contact information, such as an email address in an email address field 268 as shown in FIG. 7. With the different inputs received regrading initial eligibility, a user can then proceed to show eligibility results by selecting display element 270 and that displays the user interface 280 of FIG. 8 in response to a determination that the entered information satisfies the minimum requirements. That is, the user interface 280 notifies the user that he or she is a candidate for disability benefits and is promoted to create an account using input fields 282 (e.g., name, email, password, etc.) and selecting a create account display element 284. It should be noted that a display element is any graphical representation or text, or combination thereof, generated and displayed as part of the user interface in various examples.

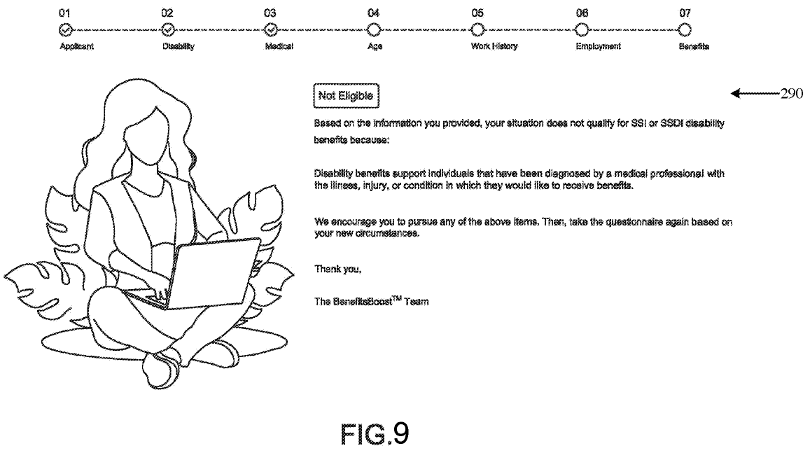

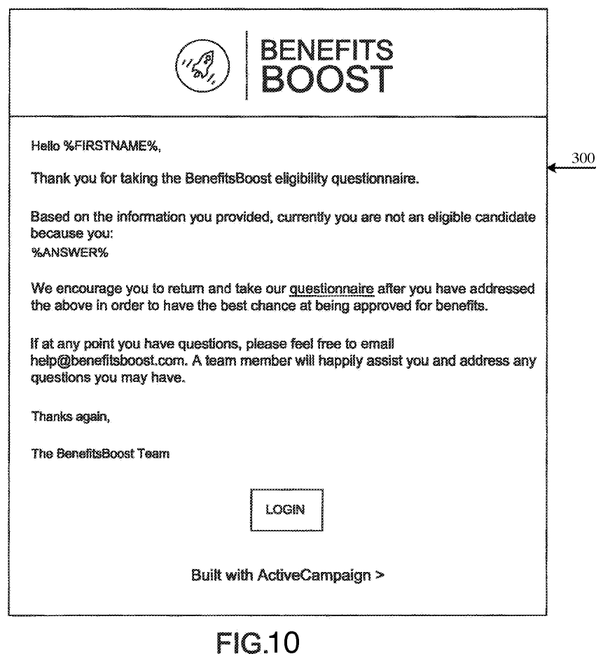

In some examples, if a determination is made that the user does not meet the minimum criteria, the disability benefits application analyzer 100 notifies to the user of each criterion that was not met, and identifies options or solutions to facilitate the user meeting the criterion, such as shown in the user interface 290 of FIG. 9. In one or more examples, an electronic communication 300 as illustrated in FIG. 10 is triggered (e.g., an email communication) to any user that does not meet the minimum criteria and allows the user the opportunity to attempt to address any deficiencies in the criteria. As should be appreciated, this provides full transparency to the user as to how to meet the minimum criteria. In some examples, the system is configured to transmit subsequent electronic communications, such as reminder communications in the event the user desires to update his or her information to address any criteria deficiencies.

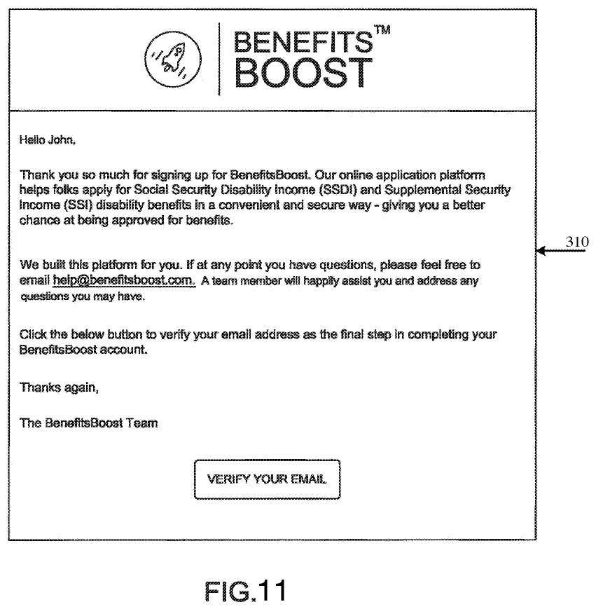

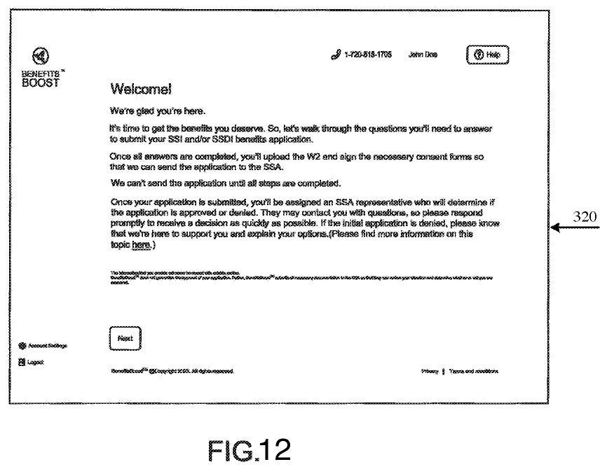

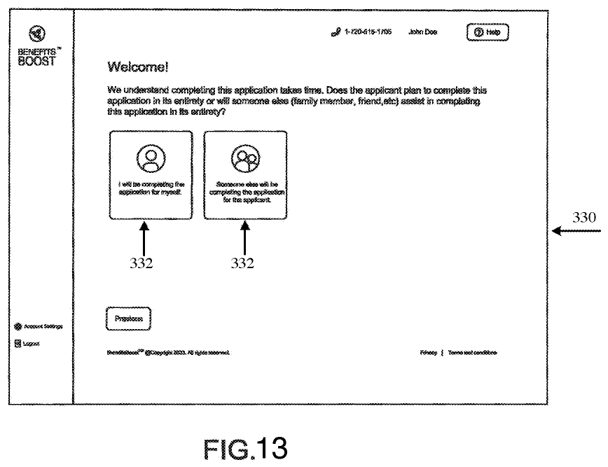

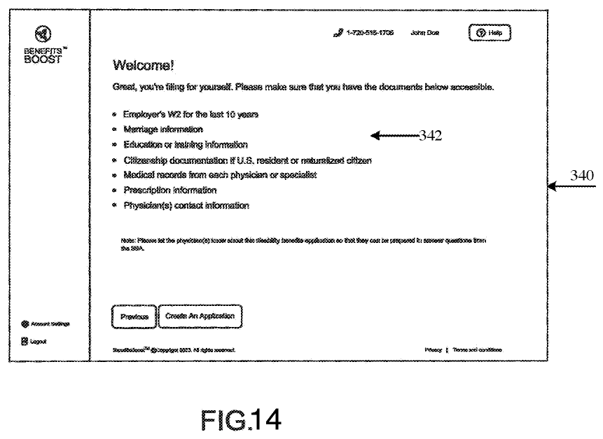

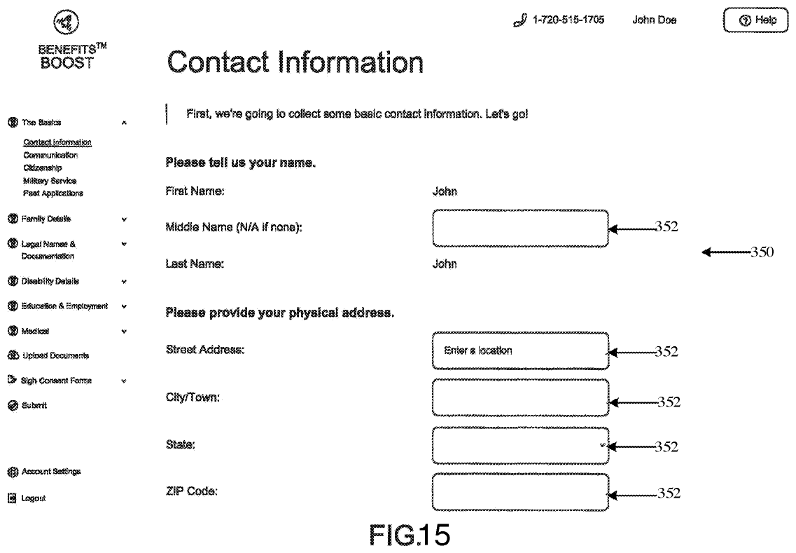

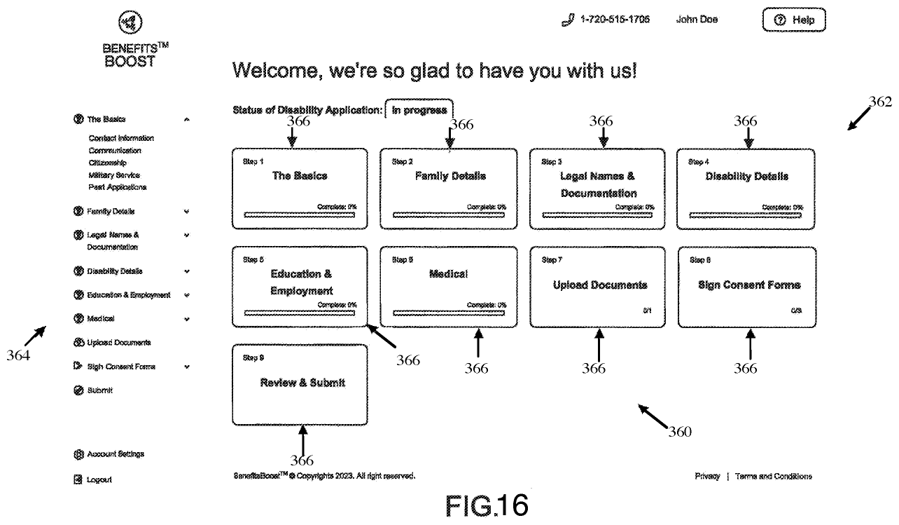

Similarly, if the user does meet the minimum criteria, the disability benefits application analyzer 100 notifies to the user, and prompts the user to verify information as the final step of the account creation process, such as shown in the electronic communication 310 of FIG. 11. Thereafter, a user interface 320 as shown in FIG. 12 is generated and displayed to complete the application process. It should be noted that if a user that initially did not meet one or more criterion subsequently updates responses based on suggestions as described herein (e.g., a technical error or discrepancy in entering one or more responses), the user can create an account and will be guided through the remainder of the application process as described herein. In various examples, the account-based portal uses multi-step verification or authentication, which can be any suitable type of process. It should be noted that the user in some examples is the applicant and in other examples can be a representative of the applicant, such as an attorney (using the herein described systems as entry for the initial application process). For example, as illustrated by the user interface 330, user selectable elements 332 allow for identifying a user applying for themselves or for someone else.

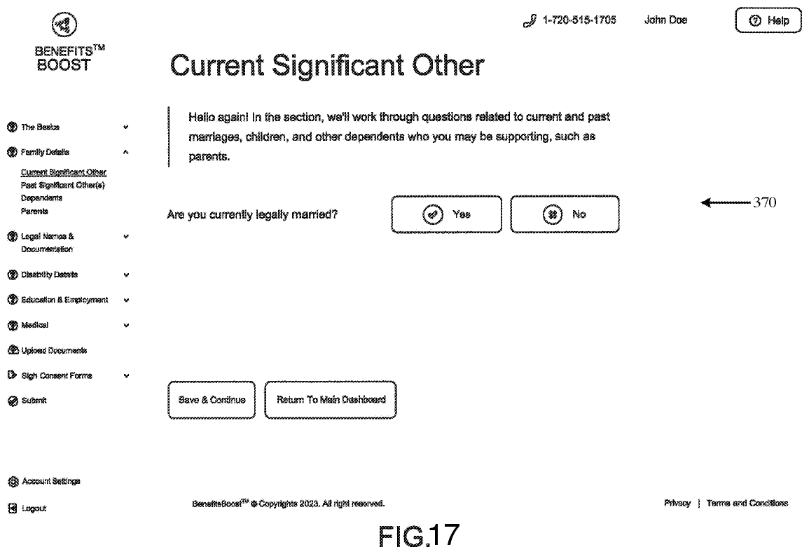

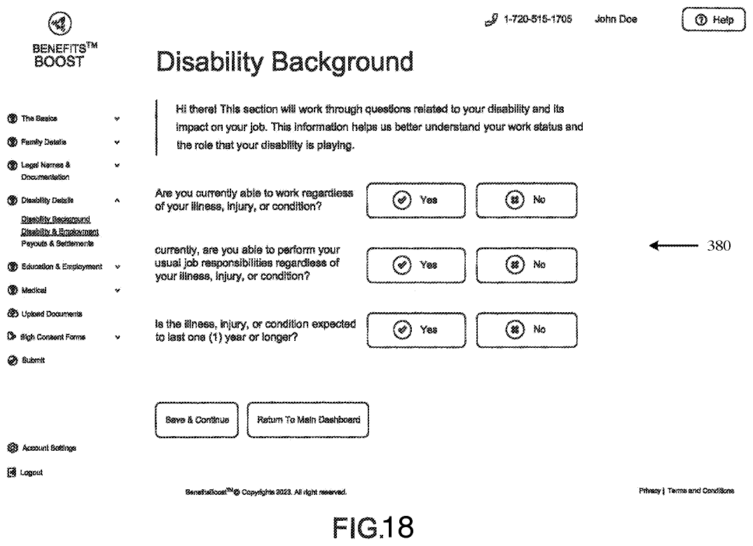

In the instance where a user is applying for themselves, a user interface 340 is display that provides a list 342 of documents or information that will be needed to complete the application process, such as financial or wage documents, marriage information, education or training information, citizenship information, medical records, prescription information, physician information, etc. Different user interfaces 350, 360 as shown in FIGS. 15 and 16 are then displayed to facilitate guiding the user through the application process. For example, user interface 350 illustrates user input fields 352 (which may include drop-down menus) that allow the system to receive user inputs related to address, etc. User interface 360 illustrates a dashboard 362 that is provided in various examples that identifies and outlines the various steps of the process, as well as indicates progress in each of the steps, as well as the status of the application. In one or more examples, a question hierarchy 364 is provided that shows status within a particular section 366 of the application process. It should be noted that the user can “jump” between different sections 366 and complete or a portion of each of the sections 366 in any desired order.

Thus, as described herein, and in some examples, upon entering the platform for the first time, the platform surveys customers to determine what records they have physically or digitally available and what records they do not have access to. The platform prompts the customer to gather the remaining documents they do not have access to. This information is used to guide further education on the website regarding the records that should be proactively gathered. This feature helps set expectations and clarity as to what documentation is helpful to fill out a disability benefits application. Additionally, new users are prompted with a self-guided (optional) tour that shows the users the main features of the platform so they become familiar with the main dashboard 362 quickly. In some examples, before a customer begins answering questions, the customers are given the opportunity to be guided by a live disability expert. This is an added service that the customer can choose to purchase in some examples. The guided disability expert is a service configured is some examples for those that are less confident in their technical abilities or have an injury, illness, or condition that prevent them from completing the application on their own.

In operation, before a customer logs out, the platform asks “Are you sure you want to leave?” If yes, the platform follows up to understand why and when they anticipate being back. This only occurs after the first and third time the customer wants to log out of the portal in some examples, but can occur at other times. This gives insight into why customers stop along the way and how to help them finish the application, which can be used as part of a learning process (e.g., machine learning in some examples). Also, in some examples, as customers answer each question, the platform gives customers the ability to inform one or more experts or advisors that they do not know how to answer a specific question. For example, the customer has the ability to select “Help, I need further direction on how to best answer this question for my situation.” The customer and the question that they cannot answer is sent to an expert or advisor so they can help resolve the issue. This enables the quick resolution of issues or roadblocks for customers.

With respect to the sections 366, all required questions have to be answered before the section or step is completed in some examples. AIs Question and Answer Sections, Upload Documents Section, and Signing Consent Forms Section have to be completed before the customer can proceed to the last sections: Reviewing Application and Submitting Application in some examples. As described herein, the platform includes a status of the customer's application, and the status updates as the application moves through the initial application process. This feature keeps customers updated on advancements of their application. The platform triggers an email communication when a status changes so customers know to log into the platform to see the update. The statuses in some examples include: Application in Progress, Submitted Application, Application in Review, SSA Application Decision: Approved or SSA Application Decision: Denied.

It should be noted that the main dashboard 362 is displayed differently for attorneys/law firms. Prior to seeing the graphical tiles representing the sections 366 to execute for a particular application, the attorney is provided the option to start a new application for a client as well as a list of active applications that have yet to be submitted to the SSA. The attorney can select (e.g., click) into any one or the active applications to continue filling out the application on behalf of their client. Additionally, an attorney or law firm is provided additional information, such as the number of active applications, number of submitted applications, number of approved applications, number or denied applications, and percentage of approved applications compared to the total submitted. Attorneys have the ability to remove/delete an active application in various examples.

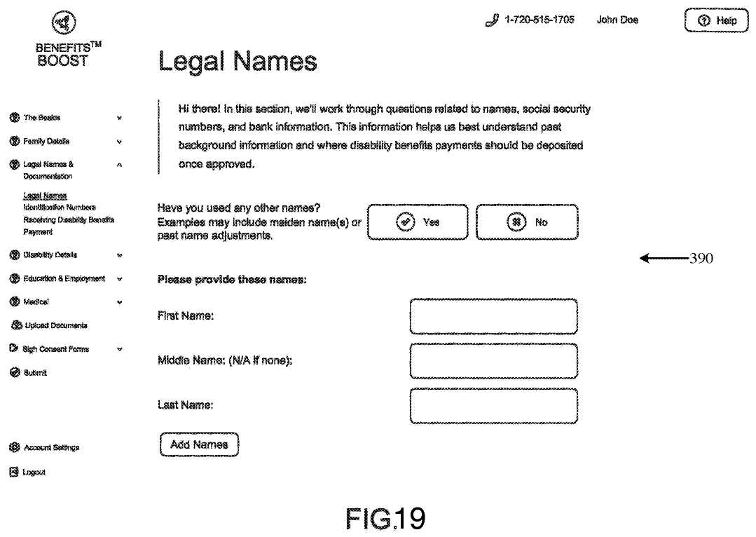

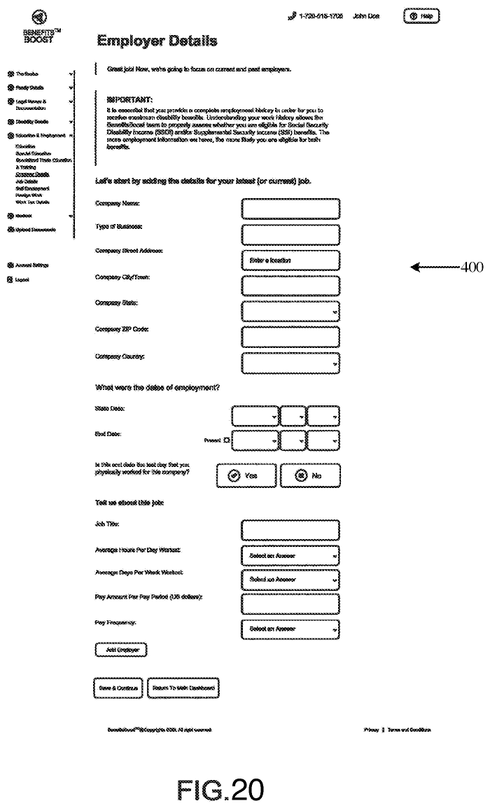

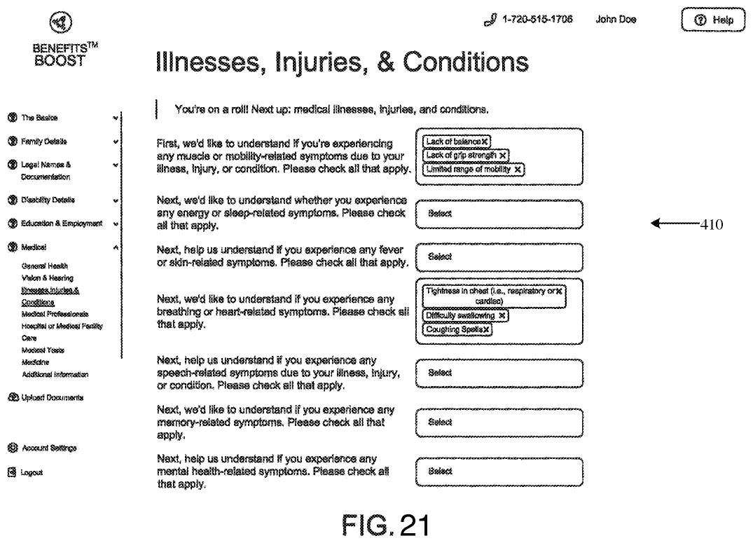

Thus, the platform breaks down the questions to be answered within a disability benefits application into “bite-size”, “digestible” questions written for non-experts. The questions are presented in an engaging manner to encourage continuation in the process. The platform has a customized experience for both an individual that is applying for themselves as well as someone who is applying for someone else (family member, friend, or organization member). This allows the platform to prompt customers in the correct manner or context. One example includes using their name when communicating instructions. This also provides an elevated level of customer service and user experience to the individual filling out the application. In some examples, user interfaces 370, 380, 390, 400, 410 as shown in FIGS. 17-21 are configured to receive user information relating to the application, including significant other information, disability background information, legal name information, employer detail information, and medical information (e.g., illnesses, injuries and conditions information). It should be noted that the interfaces 370, 380, 390, 400, 410 are merely examples of the information that can be collected and analyzed. The interfaces 370, 380, 390, 400, 410 are also merely examples of user input elements, fields, etc. that can be presented to the user. The format, configuration, arrangement, etc. of the interfaces 370, 380, 390, 400, 410 can be varied as desired or needed.

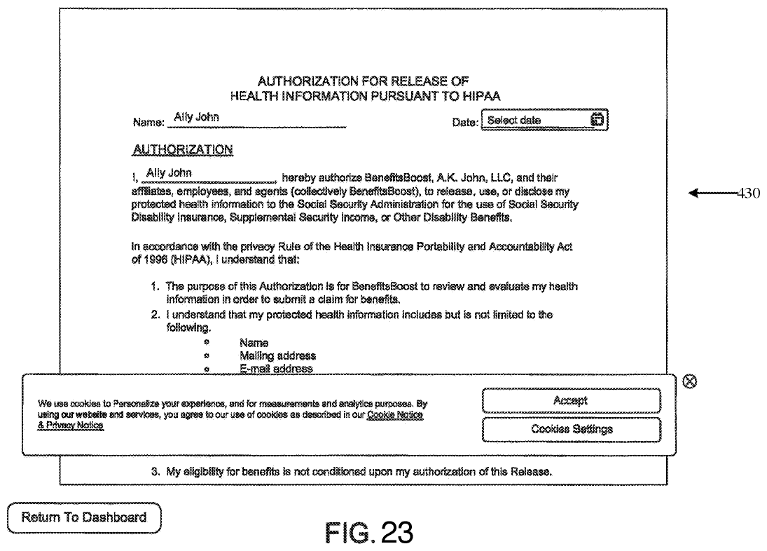

It should be noted at different points in the process, the user can provide different required or desired information. For example, prior to answering employment-related questions, customers have the opportunity to provide their social security number. The platform then retrieves the customer's W2 employment documentation in some examples. This retrieval in some examples is implemented using an API connection to an authorized third-party tool to retrieve the employment document the customer provided consent to prior to using this feature. In one process flow, customers confirm each W2 is a part of their work history to ensure someone else's information is not tied to their application. This feature provides customers an improved user-experience so the customer can answer the questions accurately and quickly, giving the customer a greater chance of approval and saving the customer time and effort. It should be noted that any type of authorization can be implemented according to the present disclosure, such as the user interface 430 illustrated in FIG. 23 that allows for an electronic HIPAA authorization.

In some examples, the system is configured to allow customers to review and sign each consent form one at a time. The platform generates a consent form with each customer's information within the required fields of the consent form based on previous answers the customer voluntarily provided within the platform. This provides a more convenient and efficient user-experience so customers do not have to enter their information twice. Through an integrated third-party contract signature tool, such as DocuSign or a similar service, the platform allows each customer to review the filled-out consent form, date, and sign each consent form. The integrated third-party contract signature tool gathers handwritten signatures from each customer, which is the only signature option in some examples. The third-party tool obtains handwritten signatures by requiring each customer to upload a scanned document, digital document or image of the applicant's handwritten signature. The third-party tool applies the handwritten signature to the consent form the customer is reviewing and signing. In addition, the platform requires an image be taken of the applicant at the time the signature is applied to the consent form to ensure further authenticity in some examples. These features ensure a seamless experience because customers are staying within the experience, avoiding adding duplicate information, and signing each consent form with multiple levels of verification.

The platform allows customers to review all or their fully completed application answers and documentation to ensure accuracy or all the information within their application. Customers have the ability to return to any previous sections to edit their answers or employment documentation. If desired, one or more examples allow the user to edit any information, with the platform saving the new information.

In some examples, one or more implementations of the platform are configured to allow customers to view added services that can further improve the likelihood for approval. This ensures each customer has the opportunity to consult with a disability expert until the customer is ready to submit the application. These services include, for example, expert verification, full-service one on one consultation, digital storage for all benefits-related documents, among others. In some examples, these services are pay add-ons with a payment card, through a third-party transactional service, or other payment service. This ensures the user-experience is easy and convenient. If the added service the customer has purchased includes a consultation or appointment of any kind, the customer is prompted to select a meeting date and time the customer would like to speak with a disability expert.

The internal platform in some examples is accessed by administrators and partners in some examples. This internal platform allows for different levels of access to the capabilities (as described in the workflows herein). In some examples, a dashboard shows all applications that have been started, all applications that have been submitted, all applications that have purchased an added service, and all applications that need to be reviewed by a disability expert because the applicant selected an added service that requires a an individual to execute the service. The internal platform includes all reporting and data views.

In some examples, analytics are acquired at different steps throughout the process or workflow. The platform identifies, for example, areas where customers are spending time and/or stopping in the workflow. These areas are re-evaluated to make the areas easier to understand or accomplish for the customer in some examples. This ensures the user experience continues to evolve and satisfy customers.

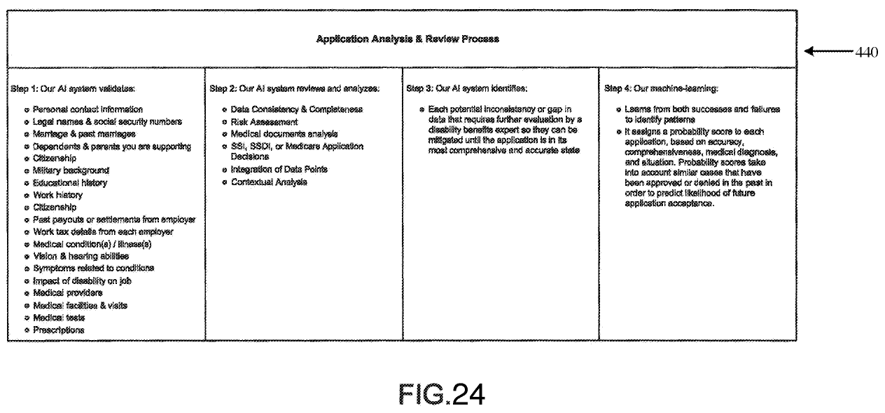

In some examples, the internal platform digitally reviews each application for accuracy using an application an analysis review process 440 as illustrated in FIG. 24. In some examples, the platform of system is configured to scan the application answers and documentation to identify any mismatched or concerning information. If an answer or series of answers is flagged, the internal platform pulls the application into a separate queue to be reviewed.

In some examples, the platform collects volume and tine spent data for one or more of the following:
1. Individuals that meet the minimum requirements;
2 Individuals that do not meet the minimum requirements;
3. Individuals that create an account;
4. Individuals that stop before finishing creating an account;
5. Individuals that are actively filling out an application;
6. Individuals that have started each section;
7. Individuals that have completed each section;
8. Individuals that have submitted an application;
9. Individuals that are awaiting a decision by the SSA;
10. Individuals that have had their initial application approved;
11. Individuals that have had their initial application denied;
12 Individuals that have provided an NPS score; and
13. Individuals that have provided a rating and/or review.

In some examples, the platform collects the percentage of time each answer option is selected by customers. The platform compares answer options to applications that are approved or denied to understand (e.g., machine learn) if there is a higher probability of an application being approved based on answer options related to: symptoms, diagnoses, medical professionals, employment history, and familial situations. The platform utilizes AI and machine-learning databases in some examples to learn over time the likelihood or probability of an application being approved based on the given answers by a customer in combination with historical data (e.g., data relating to previously submitted applications). That information is shared with customers to better inform decisions and provide transparency.

In some examples, the system also acquires logged in time per visit by each customer to understand how long the process takes within the platform. This allows for monitoring how improvements in the platform continue to decrease the amount of time it takes to complete the process (increasing customer satisfaction).

In some examples, the system acquires the average length of time an individual takes to complete their initial disability benefits application. This data allows for an understanding of the likelihood he or she completes the application based on the number of days their application has been in progress or active. This data also identifies key times the system is to be communicating to customers to ensure the customers complete their application.

In some examples, the system acquires the number of total applications, number of total active applications, number of submitted applications, number of approved applications, number of denied applications, and the percentage of approved applications compared to the total for each attorney/law firm account. This ensures each attorney/law firm account abides by the terms set forth and a minimum percentage of approved benefits, such as based on a defined amount.

In some examples, the system acquires the total number of applications, number of total active applications, number of submitted applications, number of approved applications, number of denied applications, and the percentage of approved applications for al attorney/law firm accounts. The system also acquires the number of accounts 25% complete, 50% complete, and 75% complete by all attorney/law firm accounts, or other percentages.

In operation, in some examples, after customers have answered both the symptoms and medical professional questions, the platform identifies symptoms, illnesses, or injuries that do not have a corresponding medical professional that was provided by the applicant (e.g., identifying information that was left out by the applicant). This is performed in one or more examples by mapping medical professional procedures, treatments, specialties, illnesses, etc. that each profession handles or is associated with. If the platform identifies a symptom, injury, or illness where a medical professional was not supplied by the applicant, the platform prompts the applicant to provide the medical professional information for that symptom, injury, or illness so there are no gaps (or incomplete information) in the application. This process ensures accuracy and provides the greatest likelihood or being approved for benefits in various examples.

In some examples, the platform identifies commonly related injuries, illnesses, and conditions based on the medical conditions, illnesses, or injuries the customer voluntarily provides within their application. The platforms queries the customers if the customer experiences any of those commonly related injuries, illnesses, or conditions to ensure that the customer is not leaving anything out of the application. These features also provide customers an improved user experience so that the customer can answer the questions accurately and quickly, resulting in a greater chance of approval and saving time and effort.

As such, one or more features provide customers an improved user experience to answer the questions accurately and quickly, thereby giving them a greater chance of approval and saving time and effort, including processing resources. As described herein, if a customer answers a question that disqualifies the customer from satisfying the minimum requirements for disability benefits, the platform provide a notification (e.g., provides a pop-up communication) that confirms this answer is correct and prompts the user to stop because the user no longer meets the minimum requirements. This process ensures the user do not spend unnecessary time completing an application when the user does not meet the minimum requirements. It should be noted that if the customer selects the help option in one of the user interfaces (e.g., in the upper right-hand side of the view), a window is displayed that allows the user to, for example, message the expert or advisor questions or concerns. This ensures customers have the option to call or email at any point in the process.

The internal platform is some examples is configured to have a queue that includes all applications that have flagged information. An application entering the queue triggers a communication to the appropriate individual there is a new application in the queue to take action on. This ensures the individual is notified and can appropriately take action. Customers that that have purchased an Expert Verification or Full-Service One on One Consultation skip this queue and are entered into their appropriate queues for those services in some examples. Flagged information is still carried through so the information can be viewed.

An individual can check out an application from the queue show others that the individual is executing the review. Checking out the application automatically adds this individual's name to the SSA-1696 form as the specified representative for this applicant in some examples. When the application is checked out, the internal platform shows all of the completed application answers and documentation in an easy-to-read format that makes the review process faster. For example, this view includes flagged information and contact information. The individual can deem the flagged information acceptable/passes or not acceptable/fails. The individual can contact the customer by phone or email to ensure the information is accurate and edit the information with the appropriate permission. The individual works with customers until the flagged information is acceptable/passes in some examples. The internal platform in is configured to allow individuals to enter comments along with an application or account to provide full context regarding the review situation or decision. After the review has finished, the individual is able mark this step completed. This enables the application to move to the next step.

The internal platform allows the individual (e.g., disability representative) to adjust the application type for the applicant to ensure they are applying for the correct disability benefits based on the information she or he provided within the application. The individual has the ability to identify the application type as an SSI application, SSOI application, or SSI and SSDI application in some examples.

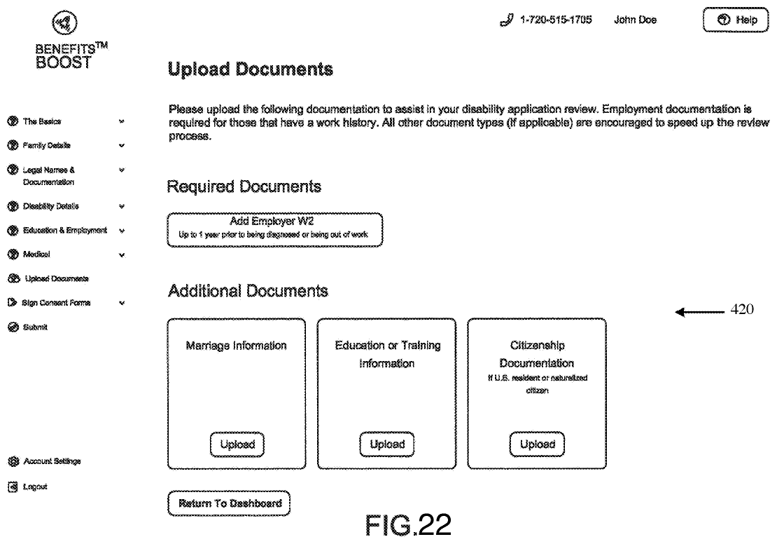

In some examples, a user interface 420 as shown in FIG. 22 is configured allow user input of documents. For example, the user interface 420 allows for uploading of one or more documents in some examples to use as part of the disability application submission. For example, the platform is configured to allow customers to upload scanned documents, digital documents, or take an image of their W2 documentation pertaining to their employment up to the last 10 years, or other documentation. Customers have the opportunity to upload multiple W2s or documents. Alternatively, customers can provide a social security number during this step. The platform retrieves their W2 employment documentation as described in more detail herein. These features provide customers with a seamless experience by staying within the platform, easily uploading or adding documentation to their application for greater approval chance, and continuing the overall process quickly, saving time and effort.

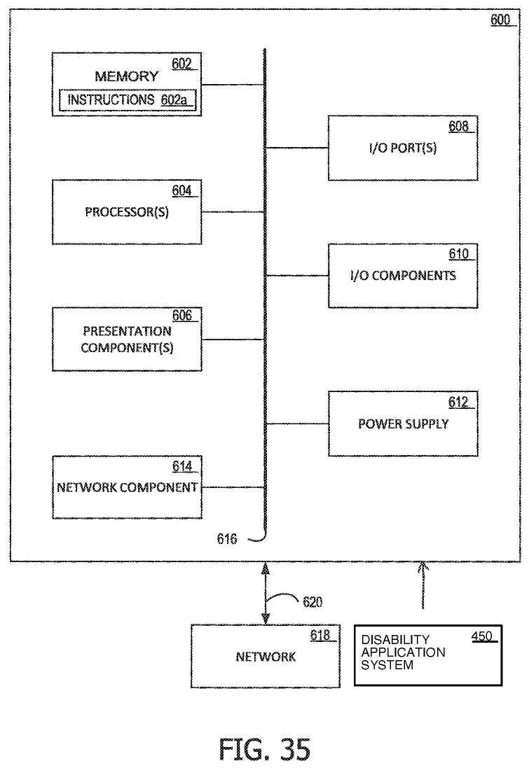

Once completed, the platform is configured to submit the final applications to the SSA or other disability program through a digital service or connection, such as an API or through an FTP server. For example, as illustrated in FIG. 35 the final applications are submitted to a disability application system 450. This ensures applications are submitted promptly without delay to shorten the waiting period for customers. The applications are configured to be submitted in an optimal format set forth by the SSA or to comply with other system requirements. This ensures that the applications are in compliance and provided to the SSA in the easiest format for review.

The platform in various examples is configured to allow printing of all signed documentation to send along documentation to the SSA via fax in addition to sending each full application digitally to the SSA in some examples. This is to ensure the local SSA office receives the appropriate cover letter, medical consent form, and SSA-1696 form, etc. for each application.

The platform in some examples triggers a communication to an affiliated partner that can assist the customers in next steps of appealing the initial application decision if denied. This allows for a determination of possible next steps to obtain the benefits. Affiliated partners in some examples are select disability benefits attorneys, firms, or representatives that meet the standards of customer service, counsel, and communication, in some examples. Customers are also able to leave feedback and reviews using any suitable review and rating mechanism.

Thus, various examples provide an improved process of disability benefits submissions and applications. For example, the platform includes multiple accessibility features throughout the user experience that include, for example, audio for questions and answer options, tabbed-format for keyword accessibility, higher contract, larger text, and voice command for answer options. Selecting any answer option automatically saves the selected answer to reduce “clicks” for customers. Answers are saved even if the customer exits a particular section 366.

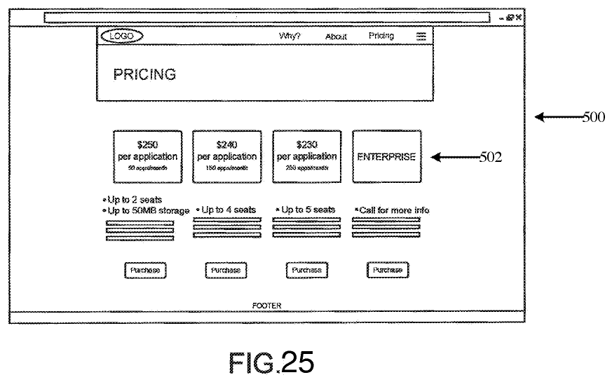

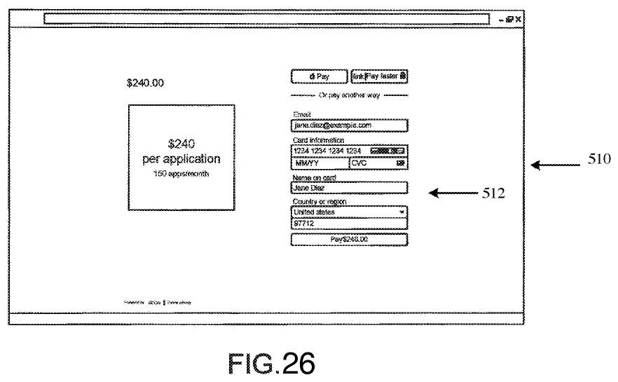

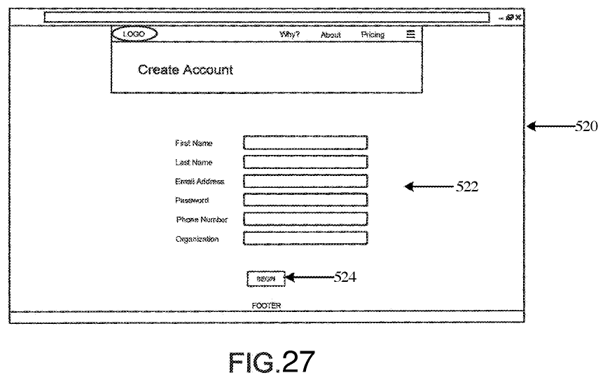

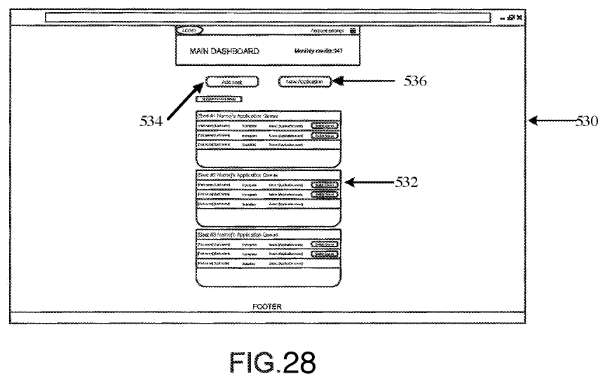

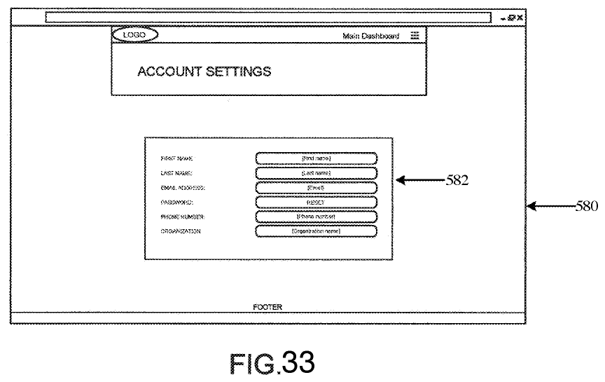

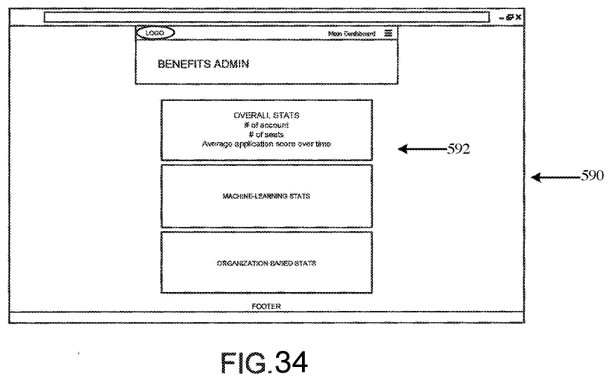

It should be noted that while one or more here described implementations relate to a B2C application, one or more implementations can be configured for a B2B application, or other application. For example, FIGS. 25-34 illustrate user interfaces configured for a B2B application. In particular, FIG. 25 illustrates a user interface 500 that displays graphical representations 502 (e.g., pricing for multiple applications) that are provided for use by professionals, such as paralegals, representatives, and attorneys specializing in disability benefits. In some examples, the user interface 500 is configured as a pricing page offering multiple package tiers. These packages vary based on the number of applications and seats, accommodating organizations with multiple paralegals. Users can easily select a suitable package and proceed to purchase with a simple click. Thereafter, a user interface 510 shown in FIG. 26 allows users that select an appropriate package for purchase to establish payment using inputs 512, considering the desired number of seats and applications for utilizing the system. After entering payment details, a user interface 520 as shown in FIG. 27 that is configured to allow creation of an administration account for both the user and their organization, if applicable. In some examples, this process includes entering personal details such as first name, last name, email address, password, phone number, and organization name, etc. in fields 522. In some examples, with the system operating on an account-based model, users provide this information before gaining access to the platform. Upon creating this initial account, users can subsequently use their email address, phone number, or password for future logins and continued use of the platform within the same account. After completing these details, users will activate a ‘Begin’ selection element 524 to enter the platform, wherein a dashboard 532 is displayed by a user interface 530 as shown in FIG. 28.

More particularly, the dashboard 532 is configured as a primary dashboard that is the gateway for both new and returning users. A hero section displays remaining credits, diminishing with each new application initiated. On depletion, users are notified and given the option to replenish for the current month. Credits ensure fair compensation for platform usage, benefiting both users and organizations.

The platform is configured to manage the disability benefits application information collection, navigating the review process to identify gaps, inconsistencies, or opportunities for further completion as described in more detail herein. In some examples, administrative users can add seats for additional team members by providing basic information and using an Add Seat selection element 534. A notification is sent to the new member, who creates a password and an account tied to the organization. In some examples, seat addition is limited by the package, requiring custom arrangements for more seats.

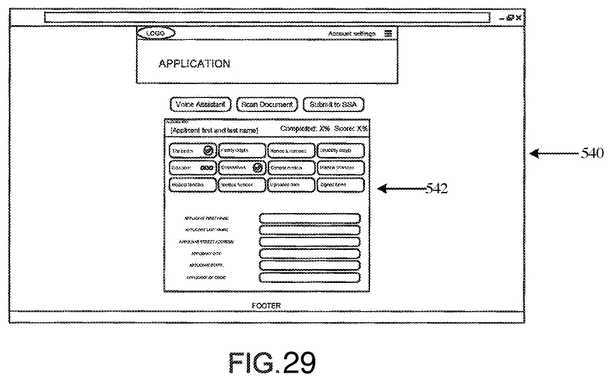

Activation or selection of a New Application selection elements 536 results in display of a user interface 540 as shown in FIG. 29. That is, upon selecting a “New Application” from the main dashboard, users initiate the completion of questions, documentation upload, and the scanning of signed consent forms for disability benefits application submission as described in more detail herein. Attorneys, paralegals, or representatives can either complete the information on behalf of their clients or send an email notification for clients to answer questions themselves in various examples.

In various examples, platform users and clients can utilize the platform's voice assistant, which is an AI-powered feature asking, following up, and clarifying questions. Spoken answers via the voice assistant are auto-filled into application fields 542 for enhanced accessibility in some examples. The AI voice assistant identifies inconsistencies and cross-checks the information provided in some examples as described in more detail herein. For example, if an individual mentions seeing Dr. Jones in St. Louis, the voice assistant supplies relevant details about Dr. Jones' clinic, seeking confirmation for accuracy. This streamlined interaction not only reduces effort for users, but also accelerates the process and improves accuracy.

In some examples, the New Application selection element 536 (e.g., New Application virtual button) initiates the information collection for a client. Below, queues for each advisor or other individual is displayed ongoing or pending applications. The status, including completion and submission to the SSA, is visible. The application score reflects completeness and highlights areas needing attention. A score approaching 100% indicates thoroughness, but submissions don't require full completion, considering unique circumstances.

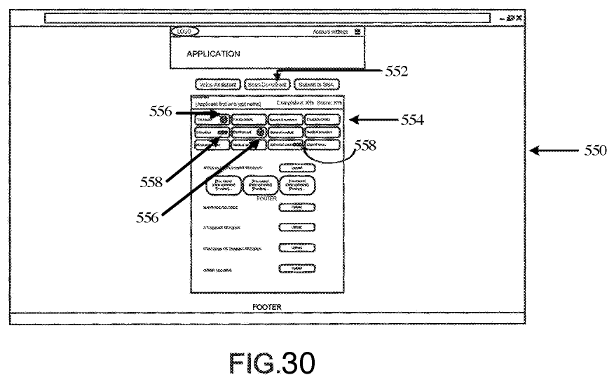

In each application, users, whether representatives, platform users, or clients, provide details like contact information, family particulars, legal names, Social Security numbers, disability specifics (including its impact on employment and education), employment details, and general medical information with the user interface 550 shown in FIG. 30 (using elements 554). The application also encompasses inquiries about medical providers, facilities, and medications. Users can upload relevant documents, including signed consent forms like Medical Consent Forms and the 1696 Representative Consent form. A “Scan Document” selection element 552 enables users to scan or upload images of medical, employment, education, marriage, identification records, or any relevant documents. The platform scans and auto-fills information into the application, streamlining the completion process. Each completed application section is marked with an indication of completion, such as a green check symbol 556, while sections in progress are indicated by a different indicator, such as ellipses 558. Users can select each section to view completed answers, and the platform displays the percentage of completion and an application score, reflecting comprehensiveness, accuracy, and any gaps or inconsistencies in some examples. Users can upload and preview documents, delete or re-upload documents, and add multiple documents for various records, offering flexibility and thorough case support for disability benefits applications submitted to the SSA

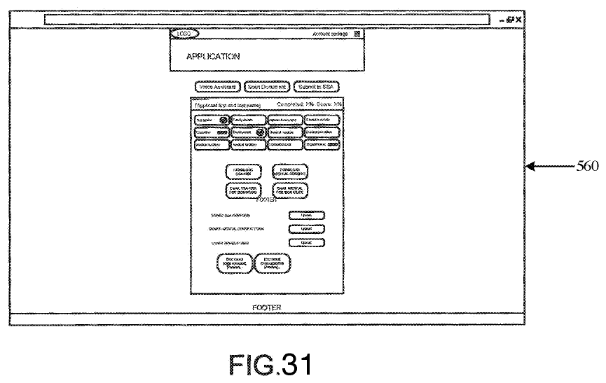

Users can download forms using the user interface 560 shown in FIG. 31, such as the SSA 1696 form and a medical consent form from the platform. The forms can be emailed to clients for electronic signatures. Upon receiving the signed documents, users can upload the signed documents to the platform, ensuring that all application-related information is centralized. Additionally, users have the flexibility to upload any other signed documentation or forms associated with the case, providing a comprehensive repository within the platform.

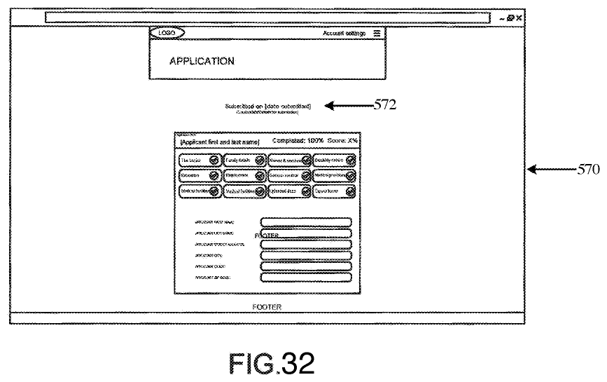

Continuing with this example, once applications reach 100% completion, users can submit through the platform using the user interface 570 shown in FIG. 32. The herein described systems and processes initiate the application via the SSA's digital portal using automated services and technology, reducing time and effort for our users. Before submission to the SSA, certified legal representative or attorney users review the application to ensure compliance with SSA guidelines. The platform displays the submission date, re-entry ID number, and details 572. However, other details can be shown. Following submission, users cannot edit information within the platform unless the application is fully resubmitted in some examples.

In various examples, a user interface 580 is configured allow viewing and modify account settings with input fields 582. For example, the input fields include first name, last name, email address, password, and phone number. However, other fields are contemplated, such as a field to receive an indication of the organization with which the user is associated. It should be noted that the organization name remains fixed and uneditable unless the user has an administrator seat.

A user interface 590 is configured as an internal administrator view that includes a dashboard 592 providing an overview of platform statistics. In some examples, the dashboard is configured to display the total number of accounts created, the aggregate number of seats across all accounts, and the average application score over time. These statistics can be further segmented by individual accounts for detailed analysis. Other statistics also can be provided as desired or needed.

In various examples, one or more examples includes AI (e.g., LegalBoost.ai) that provides AI powered and machine learning disability benefits operations. That is, some implementations include AI-powered and machine-learning technologies perform on or more of the operations or functions in the disability benefits application process as described in more detail herein. For example, the platform employs an array of validation methods to ensure data accuracy and comprehensiveness.

The AI validation methods include, for example, an address validation system utilizing Google address lookup, confirming the physical nature of provided addresses and preventing the use of P.O. boxes. The validations methods further validate phone numbers and email addresses, ensuring legitimacy. The citizenship status of U.S. residents is verified, and military-related information, such as branch, rank, and service dates, is confirmed using the validation methods in some examples.

In some examples, the review process includes an analysis of application decisions, marriage status, legal names, and other critical factors. This analysis is performed to identify patterns and potential reasons for previous denials, providing insights to enhance the quality of future applications. That is, as described herein, the system and/or platform learns based on historical data in some examples.

Moreover, the platform performs validation of employment records, education details, and medical information in some examples to ensure consistency in the provided information, identifying potential inconsistencies, and prompting users to address any disparities. The AI-driven system offers intelligent suggestions for missing information, providing guidance to users.

The technology extends validation to self-employment records, foreign work employment records, and tax details in some examples. For example, an examination of medical records, cross-referencing symptoms with diagnosed conditions, and suggesting additional symptoms that may enhance the completeness of the application is performed.

The system also validates doctors' names, medical providers' information, and appointment dates through scanned medical records in some examples, ensuring the integrity of the provided healthcare-related details. In addition, medications are validated, ensuring consistency with diagnosed conditions and suggesting any additions for completeness.

This comprehensive approach of various examples enhances the accuracy and completeness of disability benefits applications while lowering the effort and time required by disabled individuals seeking these very necessary benefits. By streamlining the application process and increasing the likelihood of approval, one or more implementations described herein contribute significantly to improving the efficiency of the disability benefits ecosystem and are designed to create a robust and reliable platform for applicants and legal professionals.

In addition, the administration area incorporates machine learning metrics, providing scores over time and predicting the percentage of applications likely to be approved or denied based on unique circumstances. Organization-specific statistics are provided to identify trends in completion rates and application scores among different entities. The machine learning insights contribute to the predictability of application outcomes, enabling a more efficient allocation of clients' and attorneys' time. This process ensures that the SSA receives more thorough, consistent, and well-prepared applications through the platform, based in part on the AI-powered Voice Assistant and the AI-driven review process as described herein.

The analysis, including the machine learning and/or AI can be performed using any machine learning or AI technology, such as using different neural networks and training methods to identify trends, suggest changes to applications, etc. In some examples, one or more outputs are generated by a computer algorithm, AI, or other type of machine learning. In some examples, machine learning is used to train or configure the disability benefits application analyzer 100 based on a training data from simulations or data received from previously generated applications to converge to more relevant application entries that increase the likelihood of approval of disability benefits. In some examples, AI is used as part of the training of and/or processing by disability benefits application analyzer 100 to generate improved applications, such as improved disability benefits applications.

With reference now to FIG. 35, a block diagram of the computing device 600 suitable for implementing various aspects of the disclosure is described (e.g., the disability benefits application analyzer 100) and that can operate in connection with, for example, the disability application system 450 to generate and submit a disability benefits application as described herein. FIG. 35 and the following discussion provide a brief, general description of a computing environment in/on which one or more or the implementations of one or more of the methods and/or system set forth herein may be implemented. The operating environment of FIG. 35 is merely an example of a suitable operating environment and is not intended to suggest any limitation as to the scope of use or functionality of the operating environment. Example computing devices include, but are not limited to, personal computers, server computers, hand-held or laptop devices, mobile devices (such as mobile phones, mobile consoles, tablets, media players, and the like), multiprocessor systems, consumer electronics, mini computers, mainframe computers, distributed computing environments that include any of the above systems or devices, and the like.

Although not required, implementations are described in the general context of “computer readable instructions” executed by one or more computing devices. Computer readable instructions may be distributed via computer readable media (discussed below). Computer readable instructions may be implemented as program modules, such as functions, objects, Application Programming Interfaces (APIs), data structures, and the like, that perform particular tasks or implement particular abstract data types. Typically, the functionality of the computer readable instructions may be combined or distributed as desired in various environments.

In some examples, the computing device 600 includes a memory 602, one or more processors 604, and one or more presentation components 606. The disclosed examples associated with the computing device 600 are practiced by a variety of computing devices, including personal computers, laptops, smart phones, mobile tablets, hand-held devices, consumer electronics, specialty computing devices, etc. Distinction is not made between such categories as “workstation,” “server,” “laptop,” “hand-held device,” etc., as all are contemplated within the scope of FIG. 35 and the references herein to a “computing device.” The disclosed examples are also practiced in distributed computing environments, where tasks are performed by remote-processing devices that are linked through a communications network. Further, while the computing device 600 is depicted as a single device, in one example, multiple computing devices work together and share the depicted device resources. For instance, in one example, the memory 602 is distributed across multiple devices, the processor(s) 604 provided are housed on different devices, and so on.

In one example, the memory 602 includes any of the computer-readable media discussed herein. In one example, the memory 602 is used to store and access instructions 602a configured to carry out the various operations disclosed herein. In some examples, the memory 602 includes computer storage media in the form of volatile and/or nonvolatile memory, removable or non-removable memory, data disks in virtual environments, or a combination thereof. In one example, the processor(s) 604 includes any quantity of processing units that read data from various entities, such as the memory 602 or input/output (I/O) components 610. Specifically, the processor(s) 604 are programmed to execute computer-executable instructions for implementing aspects of the disclosure. In one example, the instructions 602a are performed by the processor 604, by multiple processors within the computing device 600, or by a processor external to the computing device 600. In some examples, the processor(s) 604 are programmed to execute instructions such as those illustrated in the flow charts discussed herein and depicted in the accompanying drawings.

In other implementations, the computing device 600 may include additional features and/or functionality. For example, the computing device 600 may also include additional storage (e.g., removable and/or non-removable) including, but not limited to, magnetic storage, optical storage, and the like. Such additional storage is illustrated in FIG. 35 by the memory 602. In one implementation, computer readable instructions to implement one or more implementations provided herein may be in the memory 602 as described herein. The memory 602 may also store other computer readable instructions to implement an operating system, an application program and the like. Computer readable instructions may be loaded in the memory 602 for execution by the processor(s) 1204, for example.

The presentation component(s) 606 present data indications to an operator or to another device. In one example, the presentation components 606 include a display device, speaker, printing component, vibrating component, etc. One skilled in the art will understand and appreciate that computer data is presented in a number of ways, such as visually in a graphical user interface (GUI), audibly through speakers, wirelessly between the computing device 600, across a wired connection, or in other ways. In one example, the presentation component(s) 606 are not used when processes and operations are sufficiently automated that a need for human interaction is lessened or not needed. I/O ports 608 allow the computing device 600 to be logically coupled to other devices including the I/O components 610, some of which is built in. Implementations of the I/O components 610 include, for example but without limitation, a microphone, keyboard, mouse, joystick, pen, game pad, satellite dish, scanner, printer, wireless device, camera, etc.

The computing device 600 includes a bus 616 that directly or indirectly couples the following devices: the memory 602, the one or more processors 604, the one or more presentation components 606, the input/output (I/O) ports 608, the I/O components 610, a power supply 612, and a network component 614. The computing device 600 should not be interpreted as having any dependency or requirement related to any single component or combination of components illustrated therein. The bus 616 represents one or more busses (such as an address bus, data bus, or a combination thereof). Although the various blocks of FIG. 35 are shown with lines for the sake of clarity, some implementations blur functionality over various different components described herein.

The components of the computing device 600 may be connected by various interconnects. Such interconnects may include a Peripheral Component Interconnect (PCI), such as PCI Express, a Universal Serial Bus (USB), firewire (IEEE 1394), an optical bus structure, and the like. In another implementation, components of the computing device 600 may be interconnected by a network. For example, the memory 602 may be comprised of multiple physical memory units located in different physical locations interconnected by a network.

In some examples, the computing device 600 is communicatively coupled to a network 618 using the network component 614. In some examples, the network component 614 includes a network interface card and/or computer-executable instructions (e.g., a driver) for operating the network interface card. In one example, communication between the computing device 600 and other devices occurs using any protocol or mechanism over a wired or wireless connection 620. In some examples, the network component 614 is operable to communicate data over public, private, or hybrid (public and private) connections using a transfer protocol, between devices wirelessly using short range communication technologies (e.g., near-field communication (NFC), Bluetooth® branded communications, or the like), or a combination thereof.

The connection 620 may include, but is not limited to, a modem, a Network Interface Card (NIC), an integrated network interface, a radio frequency transmitter/receiver, an infrared port, a USB connection or other interfaces for connecting the computing device 600 to other computing devices. The connection 620 may transmit and/or receive communication media.

Although described in connection with the computing device 600, examples of the disclosure are capable of implementation with numerous other general-purpose or special-purpose computing system environments, configurations, or devices. Implementations of well-known computing systems, environments, and/or configurations that are suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, VR devices, holographic device, and the like. Such systems or devices accept input from the user in any way, including from input devices such as a keyboard or pointing device, via gesture input, proximity input (such as by hovering), and/or via voice input.

Implementations of the disclosure are described in the general context of computer-executable instructions, such as program modules, executed by one or more computers or other devices in software, firmware, hardware, or a combination thereof. In one example, the computer-executable instructions are organized into one or more computer-executable components or modules. Generally, program modules include, but are not limited to, routines, programs, objects, components, and data structures that perform particular tasks or implement particular abstract data types. In one example, aspects of the disclosure are implemented with any number and organization of such components or modules. For example, aspects of the disclosure are not limited to the specific computer-executable instructions or the specific components or modules illustrated in the figures and described herein. Other examples of the disclosure include different computer-executable instructions or components having more or less functionality than illustrated and described herein. In implementations involving a general-purpose computer, aspects of the disclosure transform the general-purpose computer into a special-purpose computing device when configured to execute the instructions described herein.

By way of example and not limitation, computer readable media comprises computer storage media and communication media. Computer storage media include volatile and nonvolatile, removable, and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals per se. In one example, computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium used to store information for access by a computing device. In contrast, communication media typically embody computer readable instructions, data structures, program modules, or the like in a modulated data signal such as a carrier wave or other transport mechanism and include any information delivery media.

While various spatial and directional terms, including but not limited to top, bottom, lower, mid, lateral, horizontal, vertical, front and the like are used to describe the present disclosure, it is understood that such terms are merely used with respect to the orientations shown in the drawings. The orientations can be inverted, rotated, or otherwise changed, such that an upper portion is a lower portion, and vice versa, horizontal becomes vertical, and the like.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

Various operations of implementations are provided herein. In one implementation, one or more of the operations described may constitute computer readable instructions stored on one or more computer readable media, which if executed by a computing device, will cause the computing device to perform the operations described. The order in which some or all of the operations are described should not be construed as to imply that these operations are necessarily order dependent. Alternative ordering will be appreciated by one skilled in the art having the benefit of this description. Further, it will be understood that not all operations are necessarily present in each implementation provided herein.

Any range or value given herein can be extended or altered without losing the effect sought, as will be apparent to the skilled person.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

As used in this application, the terms “component,” “module,” “system,” “interface,” and the like are generally intended to refer to a computer-related entity, either hardware, a combination of hardware and software, software, or software in execution. For example, a component may be, but is not limited to being, a process running on a processor, a processor, an object, an executable, a thread of execution, a program and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device, carrier or media. Of course, those skilled in the art will recognize many modifications may be made to this configuration without departing from the scope or spirit of the claimed subject matter.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A system for generating a disability benefits application, comprising:
a data storage device storing historical data relating to a plurality of previously submitted disability benefits applications;
a processor; and
a non-transitory computer-readable medium storing instructions that are operative upon execution by the processor to:
receive inputs relating to a disability benefits application to be submitted;
process the inputs to generate application data for a disability benefits application;
access the historical data relating to the plurality of previously submitted disability benefits applications;
update the application data based on the historical data to generate updated application data, wherein the updated application data has a greater likelihood of approval than the application data;
generate a disability benefits application using the updated application data; and
transmit the disability benefits application to a disability application system for processing disability benefits.

2. The system of claim 1, further comprising:
a user interface, wherein the instructions are further operative to:
generate one or more graphical representations including the updated application data via the user interface device.

3. The system of claim 1, wherein the instructions are further operative to:
generate one or more suggestions used to generate the updated application data, wherein the updated application data is generated based at least in part in response to a user input corresponding to the one or more suggestions.

4. The system of claim 1, wherein the instructions are further operative to:
perform machine learning using the historical data to identify one or more changes to the application data to generate the updated application data.

5. The system of claim 1, wherein the instructions are further operative to:
use artificial intelligence to validate the application data.

6. The system of claim 1, wherein the instructions are further operative to:
use one of machine learning or artificial intelligence to identify one or more patterns in the historical data and potential reasons for previous denials of one or more of the plurality of previously submitted disability benefits applications.

7. The system of claim 1, wherein the instructions are further operative to:
identify one or more potential inconsistences or disparities in one or more user inputs corresponding to the application data based on a comparison of with one or more medical records cross-referencing symptoms with diagnosed conditions.

8. The system of claim 7, wherein the instructions are further operative to:
suggest one or more additional symptoms based on the comparison.

9. The system of claim 1, further comprising:
a user interface, wherein the instructions are further operative to:
generate one or more graphical representations corresponding to a business-to-consumer application.

10. The system of claim 1, further comprising:
a user interface, wherein the instructions are further operative to:
generate one or more graphical representations corresponding to a business-to-business application.

11. A computerized method for generating a disability benefits application, the computerized method comprising:
receiving inputs relating to a disability benefits application to be submitted;
processing the inputs to generate application data for a disability benefits application;
accessing the historical data relating to the plurality of previously submitted disability benefits applications;
updating the application data based on the historical data to generate updated application data; and
generating via a user interface, a graphical representation including the updated application data, the updated application data comprising data having a greater likelihood of approval than the application data.

12. The computerized method of claim 11, further comprising:
generating a disability benefits application using the updated application data; and
transmitting the disability benefits application to a disability application system for processing disability benefits.

13. The computerized method of claim 11, further comprising:
generating one or more suggestions used to generate the updated application data, wherein the updated application data is generated based at least in part in response to a user input corresponding to the one or more suggestions.

14. The computerized method of claim 11, further comprising:
performing machine learning using the historical data to identify one or more changes to the application data to generate the updated application data.

15. The computerized method of claim 11, further comprising:
using artificial intelligence to validate the application data.

16. The computerized method of claim 11, further comprising:
using one of machine learning or artificial intelligence to identify one or more patterns in the historical data and potential reasons for previous denials of one or more of the plurality of previously submitted disability benefits applications.

17. The computerized method of claim 11, further comprising:
identifying one or more potential inconsistences or disparities in one or more user inputs corresponding to the application data based on a comparison of with one or more medical records cross-referencing symptoms with diagnosed conditions and suggesting one or more additional symptoms based on the comparison.

18. The computerized method of claim 11, further comprising:
generate one or more graphical representations corresponding to a business-to-consumer application.

19. The computerized method of claim 11, further comprising:
generating one or more graphical representations corresponding to a business-to-business application.

20. One or more computer storage devices having computer-executable instructions stored thereon, which, upon execution by a computer, cause the computer to perform operations comprising:
receiving inputs relating to a disability benefits application to be submitted;
processing the inputs to generate application data for a disability benefits application;
accessing the historical data relating to the plurality of previously submitted disability benefits applications;
updating the application data based on the historical data to generate updated application data, wherein the updated application data has a greater likelihood of approval than the application data;
generating a disability benefits application using the updated application data; and
transmitting the disability benefits application to a disability application system for processing disability benefits.
